(19) United States
(12) Patent Application Publication
(10) Pub. No.: US 2016/0338599 A1
(43) Pub. Date: Nov. 24, 2016

(54) Synchronizing Cardiovascular Sensors for Cardiovascular Monitoring

(71) Applicant: Google, Inc., Mountain View, CA (US)

(72) Inventors: Brian Derek DeBusschere, Los Gatos, CA (US); Jeffrey L. Rogers, San Carlos, CA (US)

(21) Appl. No.: 14/720,632

(22) Filed: May 22, 2015

(51) Int. Cl. A61B 5/026; A61B 5/021; A61B 5/00; A61B 5/0265

(52) U.S. Cl. CPC A61B 5/0261; A61B 5/0265; A61B 5/02108; A61B 5/7246

(57) ABSTRACT

This document describes synchronizing cardiovascular sensors for cardiovascular monitoring, such as through sensing relevant hemodynamics understood by pulse transit times, blood pressures, pulse-wave velocities, and, in more breadth, electrical conduction properties, cardiac rhythms, thoracic impedance, ballistocardiograms and pressure-volume loops. The techniques disclosed in this document use various cardiovascular sensors to sense hemodynamics, such as skin color and skin and other organ displacement. These cardiovascular sensors require little if any risk to the patient and are simple and easy for the patient to use.

### BACKGROUND

Cardiovascular disease is the leading cause of morbidity and mortality worldwide. At the same time, this chronic disease is largely preventable. Medical science knows how to save most of these lives by removing the major risk factors of smoking, diabetes, and hypertension. In addition, many people are told just what they need to do to reduce these risk factors—stop smoking, reduce sugar intake, eat healthier, reduce alcohol intake, increase cardiovascular exercise, lose weight, and, if needed, take blood-pressure medication. Nevertheless, many people do not follow this good advice. Because of this, millions of people needlessly die from cardiovascular disease.

People do not follow this good medical advice because they think they are different, they do not want to change their behaviors that are causing the disease, or they do not know what to change in their particular case. When a physician tells them that they are at risk from heart disease because they are overweight, for example, many people know that this judgment is not necessarily specific to them—it is based on averages and demographics. So being a particular weight may not negatively affect a particular patient's heart. Further, a lack of feedback that their behavior is harming their heart results in a lack of incentive for them to change their behavior.

This lack of incentive to follow good advice can be addressed by monitoring the state of the patient's cardiovascular system over time to show trends in heart health. Hard data often motivates patients to modify their behavior, such as data indicating that their heart shows measurable signs of heart disease. Unfortunately, current methods for measuring heart health can be inconvenient, stressful, and expensive. Simple home monitor products exist for measuring heart rate and blood pressure, but long-term user compliance is a problem due to inconvenience. More advanced cardiovascular monitoring, such as heart rate variability, arterial stiffness, cardiac output, and atrial fibrillation, involve expensive and time-consuming trips to a medical facility for a skilled assessment. Because of this, only patients that demonstrate late stage symptoms of heart disease are likely to receive these tests, which is generally too late to make simple lifestyle changes that would avoid a chronic disease.

Another reason that people do not follow this good advice, or do not follow it for long enough to prevent heart disease, is because they do not see the benefit. When people take the advice of changing their diet and habits—which most people do not want to do—they often do not see the improvement before they lose the motivation to continue monitoring their cardiovascular status. Because of this, many people go back to their old habits only to later die of heart disease.

### SUMMARY

This document describes ways in which to sense and assess a patient's cardiovascular health, such as through relevant hemodynamics understood by heart rates, heart rate variability, cardiac arrhythmias, blood pressures, pulse-wave velocities, arterial stiffness, cardiac valve timing, thoracic fluids, ballistocardiogram force, photo-plethysmograms, blood oxygenation, and pressure-volume loops. The techniques disclosed in this document use various sensors to sense the effects of cardiovascular hemodynamics. One challenge associated with using multiple cardiovascular sensors is timing synchronization between these sensors. Without accurate time synchronizations between sensors, higher-quality and more-useful hemodynamics are difficult or impossible to calculate. Therefore, some of the techniques herein are directed to synchronizing cardiovascular sensors for cardiovascular monitoring.

Through synchronizing and other techniques described herein, blood-flow asymmetries and trends can be determined. Asymmetries may indicate a stroke or other cardiovascular disease or pressure waveforms, which may indicate cardiac abnormalities, such as atrial fibrillation. Trends can aid a patient by helping them know if the effort they are expending to improve their heart health is actually making a difference. Further, negative trends or conditions, such as cardiac irregularities or some asymmetries can be found that can spur people to improve their health or to get medical attention. By so doing, these techniques may save many people from dying of heart disease.

This summary is provided to introduce simplified concepts concerning the techniques, which are further described below in the Detailed Description. This summary is not intended to identify essential features of the claimed subject matter, nor is it intended for use in determining the scope of the claimed subject matter.

### BRIEF DESCRIPTION OF THE DRAWINGS

Embodiments of techniques and devices for sensing cardiovascular health and synchronizing cardiovascular sensors for cardiovascular monitoring are described with reference to the following drawings. The same numbers are used throughout the drawings to reference like features and components:

FIG. 1 illustrates an example environment in which the techniques can be implemented.

FIG. 2 illustrates an example computing device of FIG. 1.

FIG. 3 illustrates an example cardiovascular sensor of FIG. 1.

FIG. 4 illustrates a method for assessing hemodynamic characteristics, including determination of a pulse-wave velocity for a patient.

FIG. 5 illustrates a male patient having various regions of which images are captured by cardiovascular sensors.

FIG. 6 illustrates various circulatory distances that can be used, along with time correlations, to determine a pulse-wave velocity.

FIG. 7 illustrates a method for determining circulatory distances, such as those described in FIG. 6.

FIG. 8 illustrates a method for synchronizing cardiovascular sensors.

FIG. 9 illustrates an example sensing milieu and timing chart for cardiovascular measurements from cardiovascular sensors in the sensing milieu.

FIG. 10 illustrates a method for assessing hemodynamic characteristics based on size, volume, or location of an organ or structure of a patient.

FIG. 11 illustrates an example device embodying, or in which techniques may be implemented that assess hemodynamic characteristics using cardiovascular sensors, including synchronization of those sensors.

### DETAILED DESCRIPTION

Overview

This document describes techniques using, and devices enabling, assessment of hemodynamic characteristic using cardiovascular sensors. Through use of cardiovascular sensors a patient's skin color, body movement, heart rate, blood pressure and various other indicators can be accurately measured over time, including by comparing measurements at different regions of the patient. For example, a cardiovascular sensor can measure a color change at a patient's cheek and, based on that color change, the techniques can determine that the patient's heartbeat has produced a peak blood-pressure flow at some particular instant at the cheek. Another cardiovascular sensor can measure a pulse wave at the patient's feet for the same heartbeat, which the techniques can determine indicates a peak blood-pressure flow at some other instant. By comparing the times and distance between these regions, hemodynamic characteristics can be assessed, such as arterial stiffness, blood pressure, pulse-wave velocity, and other measurements of cardiovascular health.

In addition to assessing cardiovascular heath at some snapshot in time, the techniques may also measure trends in cardiovascular health. By way of one example, assume that a patient has a cardiovascular sensor in her bathroom that is capable of measuring color and displacement at multiple regions, such as her neck, palm, and forehead. This cardiovascular sensor measures skin color variations between or within a region, which can indicate differential blood volume to provide a photo-plethysmogram (PPG). If the patient has other cardiovascular sensors, such as one in her bathtub or mat next to her bathroom or kitchen sink, these can further aid the accuracy and robustness of the measurements. Using these sensors, assume that over the course of a new diet and exercise routine that the techniques, using data from the cardiovascular sensors, determine that her heart's stroke volume (an important measure of heart health) has improved 6% in four weeks. With this positive feedback, this patient may continue her diet and exercise routine, thereby likely reducing the chances that she will die of heart disease.

For another case, assume that the techniques determine that there is an asymmetry in blood flow within a patient's face. This asymmetry can be indicated to the patient or a medical professional sufficient to perform further testing, as asymmetry can indicate a stroke (a deadly disease that, with a fast diagnosis and treatment can save the patient's life or quality of life) or other vascular disease.

These are but a few examples in which sensing and assessing cardiovascular health, including by synchronizing cardiovascular sensors for cardiovascular monitoring, can be performed, other examples and details are provided below. This document now turns to an example environment, after which example cardiovascular sensors and methods, and an example computing system are described.

Example Environment

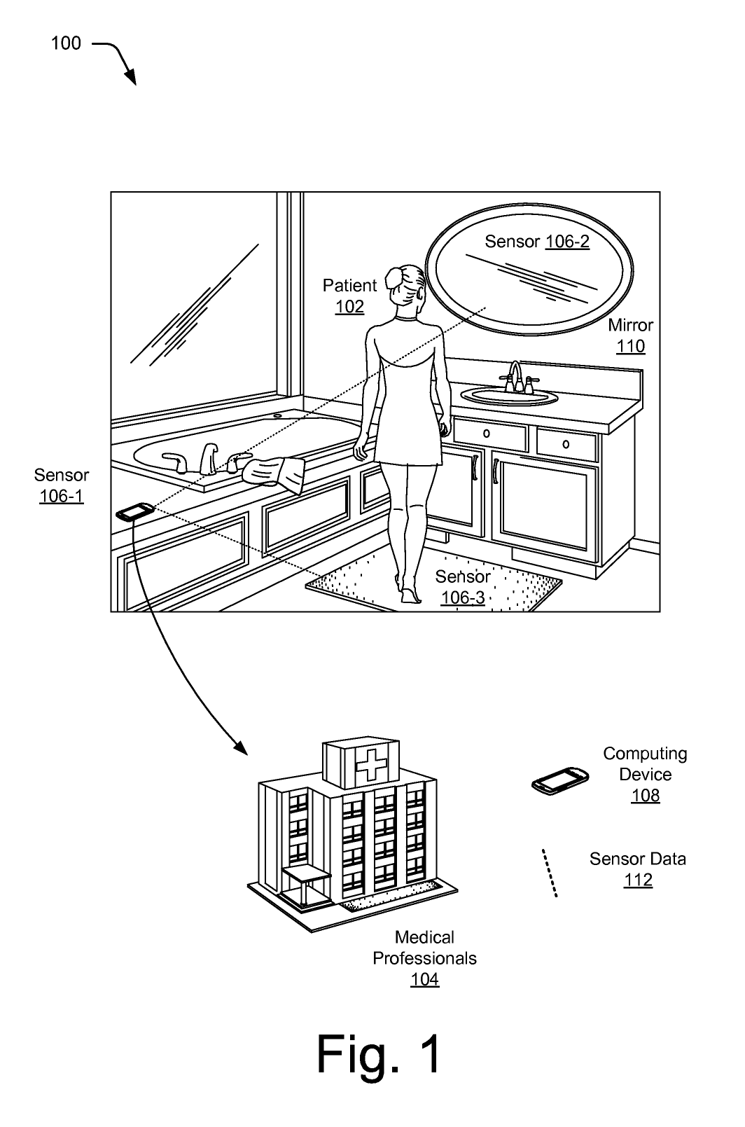

FIG. 1 is an illustration of an example environment 100 in which cardiovascular monitoring, including using time synchronization, can be employed. Environment 100 illustrates a patient 102 and a medical professional 104, family member, or other caretaker that, in some cases, will receive results of the health monitoring. This example employs cardiovascular sensors 106, a color and displacement sensor 106-1 (sensor 106-1), which is part of computing device 108, a hyperspectral sensor 106-2 (sensor 106-2), which is located within mirror 110, and a pressure and electrical-sensing mat 106-3 (sensor 106-3).

Sensor data 112 is provided by each of cardiovascular sensors 106 to some computing device. As shown, sensor data 112 is passed from sensors 106-2 and 106-3 to computing device 108 while sensor 106-1 is integral with computing device 108 and need not be passed if the techniques are performed at that device. Computing device 108 then performs some or all of the techniques, or passes that sensor data to some other computing device, such as a remote server through a communication network (not shown).

As shown with this example environment 100, a sensing milieu (e.g., cardiovascular sensors 106 in patient 102's bathroom) in which a patient lives can be used that are capable of determining a hemodynamic characteristic of a human cardiovascular system. This sensing milieu is capable of non-invasively and remotely determining this hemodynamic characteristic and trends thereof This sensing milieu senses various regions of the patient, which can then be compared, synchronized, aggregated, averaged, and so forth. These hemodynamic characteristics can be represented by cardiovascular asymmetries (e.g., due to a stoke), cardiac irregularities (e.g. atrial fibrillation), blood pressure, pulse-wave velocity, waveforms of circulating blood, photo-plethysmograms (PPG), ballistocardiograms, and pressure-volume loops, to name a few.

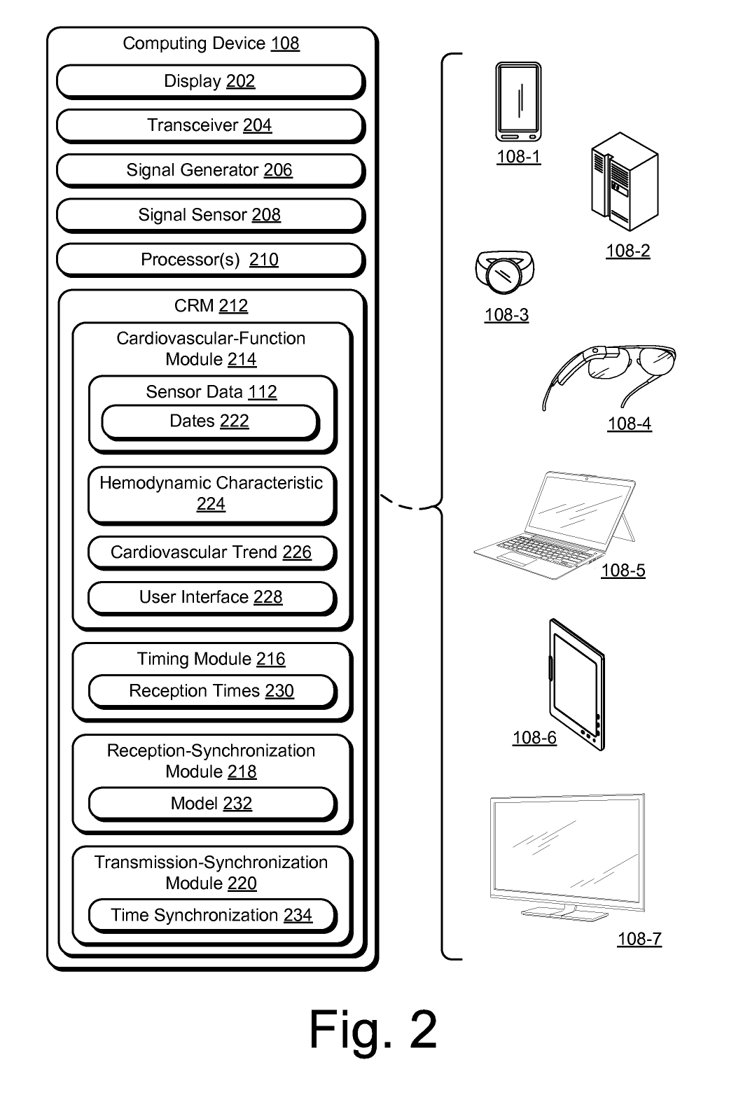

With regard to computing device 108 of FIG. 1, consider a detailed illustration in FIG. 2. Computing device 108 can be one or a combination of various devices, here illustrated with seven examples: a smartphone 108-1, a server 108-2, a computing watch 108-3, computing spectacles 108-4, a laptop 108-5, a tablet computer 108-6, and a desktop 108-7, though other computing devices and systems, such as one of cardiovascular sensors 106 that includes computing capabilities, a netbook, or a set-top box may also be used. As noted above, in some embodiments the techniques operate, in whole or in part, through a remote device such as server 108-2. In such cases, some computing can be forgone locally, e.g., through a communication device having limited computing operations or even directly from cardiovascular sensors 106 to server 108-2.

Computing device 108 includes or is able to communicate with a display 202 (six are shown in FIG. 2), a transceiver 204, an electromagnetic-spectrum signal generator 206 (signal generator 206), an electromagnetic-spectrum signal sensor 208 (signal sensor 208), one or more processors 210, and computer-readable storage media 212 (CRM 212). Transceiver 204 is capable of sending and receiving data directly or through a communication network, such as sensor data 112 from cardiovascular sensors 106 through a local area, wide area, cellular, or near-field network.

Computing device 108 includes modules enabling the computing device 108 to synchronize, whether generating or receiving, an electromagnetic-spectrum synchronization signal. Thus, computing device 108 may generate a signal, receive back a marking or indication sufficient to synchronize the various cardiovascular sensors, or receive a signal from each cardiovascular sensor and then synchronize cardiovascular measurements received with some indication of when each cardiovascular sensor transmitted the signal received by computing device 108. These cardiovascular measurements are not required to be transmitted or received quickly or all-at-once. The indication permits a later-performed synchronization of the various measurements, thereby permitting relatively low amounts of power, processing, or bandwidth to be used. Different manners for synchronizing are described below.

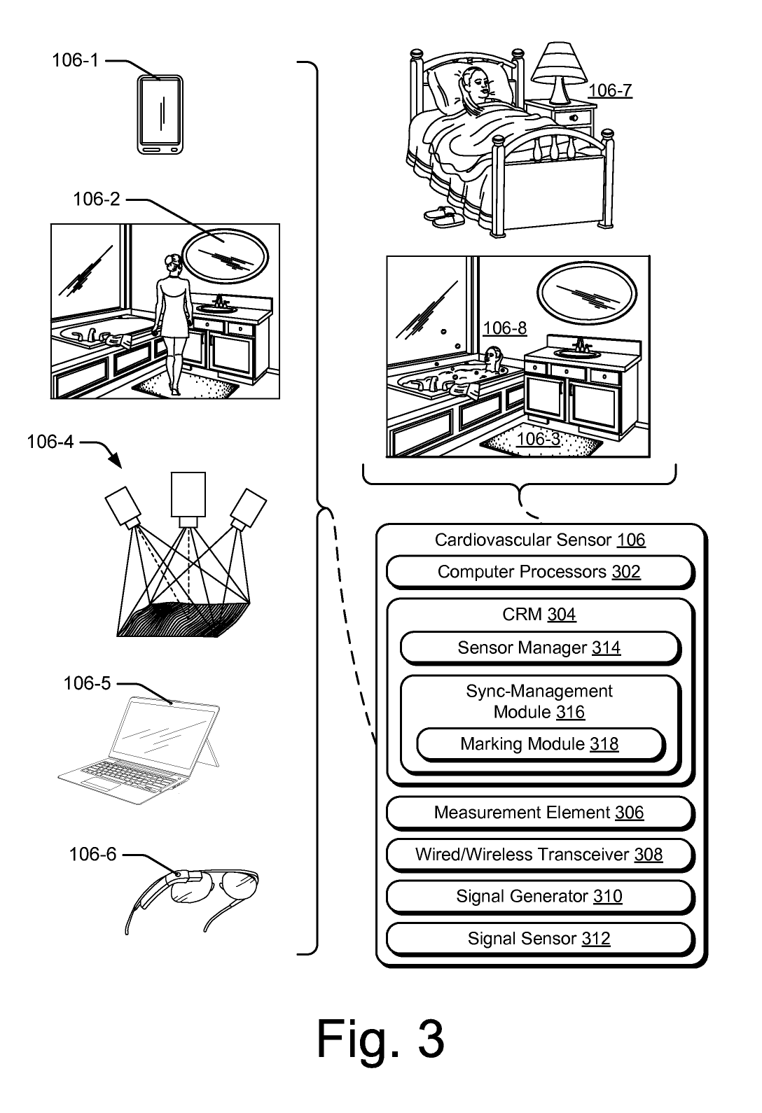

Signal generator 206 is configured to generate an electromagnetic-spectrum synchronization signal capable of capture by one or more of cardiovascular sensors 106. Signal sensor 208 is configured to capture electromagnetic-spectrum synchronization signals from cardiovascular sensors 106, such as those generally associated with or oriented to sensing patient 102. Either or both of signal generator 206 or signal sensor 208 can be usable by, or part of, computing device 108 or cardiovascular sensors 106, which will be described further below. Both signal generator 206 and signal sensor 208 (and the generator and sensor of FIG. 3) can be configured to generate or capture signals throughout the electromagnetic spectrum, such as in a radio-frequency bandwidth or an optical bandwidth to name a few. Alternatively, an audio signal (audible or inaudible to humans) could also be used. Simple examples of these include a smartphone's camera's flash as a signal generator and its signal sensor as the camera itself These signals may also include different pulse characteristics (e.g., Morse code, orthogonal coding, or different wavelengths) or contain other data by which to associate each signal with a different cardiovascular sensor 106. Signal sensors of computing device 108 or cardiovascular sensor 106 may operate in the millisecond or sub-millisecond range and have low latency. This ability aids in accurately assessing time synchronizations noted below.

CRM 212 includes cardiovascular-function module 214, timing module 216, reception-synchronization module 218, and transmission-synchronization module 220. Cardiovascular-function module 214 includes or has access to sensor data 112 from one or more of multiple cardiovascular sensors 106. Sensor data 112 can be associated with particular dates 222 for use in cardiovascular-function module 214 determining, based on a hemodynamic characteristic 224, cardiovascular trends 226. CRM 212 also includes or has access to a user interface 228, that, while not required, can be used to present determined trends, health, and medical advice to patient 102.

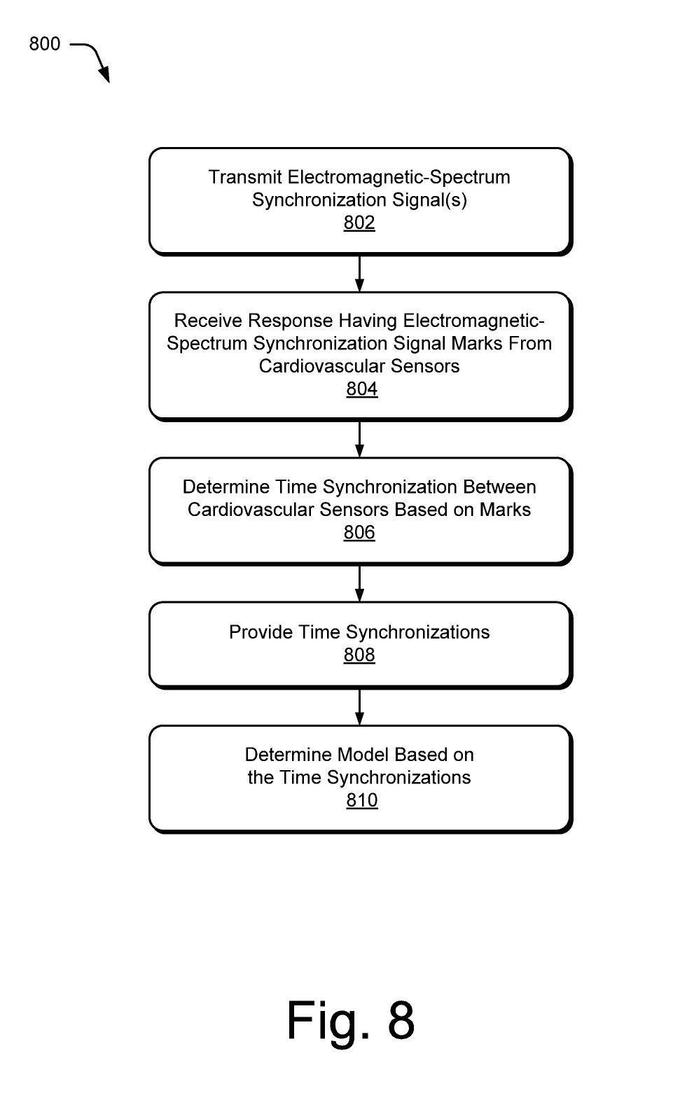

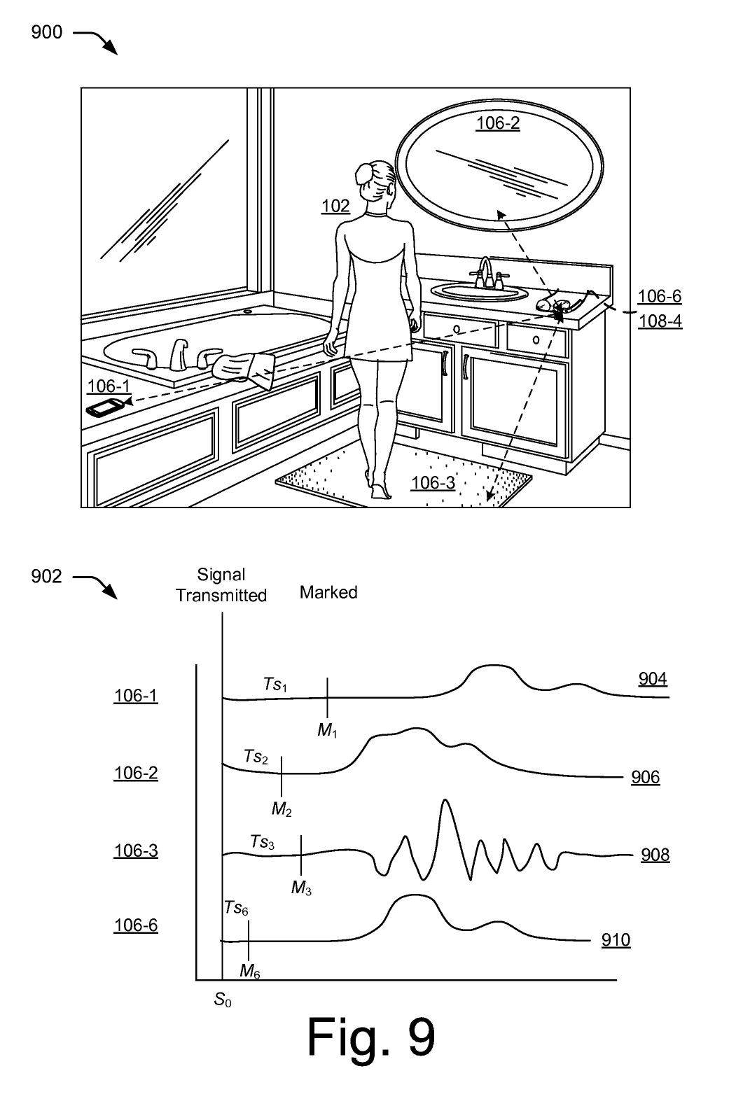

Timing module 216 is configured to precisely assign a reception time 230 to captured electromagnetic-spectrum synchronization signals received by signal sensor 208 from each of cardiovascular sensors 106. Precisely assigning a reception time includes precision of less than 100 milliseconds, though more-precise times of less than 10 or even one millisecond can be performed and is desirable in some cases. This is further described in FIGS. 8 and 9.

Reception-synchronization module 218 is configured to model, based on the precisely assigned reception times 230 for each of the captured electromagnetic-spectrum synchronization signals from timing module 216, cardiovascular timing of cardiovascular sensors 106. The model produced, illustrated as model 232, is effective to enable cardiovascular measurements from cardiovascular sensors 106 to be used to determine the cardiovascular health of patient 102, such as hemodynamic characteristics 224 and cardiovascular trends 226.

By way of example, assume that signal sensor 208 receives electromagnetic-spectrum synchronization signals from each of three different cardiovascular sensors 106 (e.g., those of environment 100). Timing module 216 may then assign reception times for each of the three signals and associate it with the sensor from which it was received. The association with the respective cardiovascular sensor 106 can be based on a characteristic of the signal being received, such as the signals having different wavelengths, amplitudes, or including data within the signal. In some cases, the association cannot be made until cardiovascular measurements are received from each of the respective cardiovascular sensors. Thus, a cardiovascular measurement for particular a particular cardiovascular sensor may indicate a time at which the cardiovascular sensor transmitted the electromagnetic-spectrum synchronization signal as well as some indication of the type of signal transmitted. This indication of the time can be an electromagnetic-spectrum synchronization signal generation mark (generation mark) at some point in the cardiovascular measurements.

Continuing the example, reception-synchronization module 218 receives the generation mark and the cardiovascular measurement from each of the cardiovascular sensors and synchronizes the cardiovascular measurements using the precisely assigned reception times and the generation mark. By so doing, each of the cardiovascular measurements can be synchronized one with the other to improve the accuracy of time correlations described below.

Transmission-synchronization module 220 manages signal generator 206 to transmit a signal to cardiovascular sensors 106 and then receives a response having an electromagnetic-spectrum synchronization-signal mark from those sensors 106. With these responses, transmission-synchronization module 220 determines a time synchronization 234 between sensors 106 based on the electromagnetic-spectrum synchronization-signal mark received from each sensor. Transmission-synchronization module 220 can then provide this time synchronization 234 effective to enable cardiovascular measurements by the cardiovascular sensors to be synchronized. As noted, this synchronization enables determination of hemodynamic characteristics of the patient that is being monitored. This is further described in FIGS. 8 and 9.

Generally, cardiovascular-function module 214 is capable of determining, based on sensor data 112, a hemodynamic characteristic of a cardiovascular system of a patient, such as patient 102 of FIG. 1. With this hemodynamic characteristic, cardiovascular-function module 214 may alert patient 102 or medical professionals 104 or family members/caretakers of a negative health condition needing immediate care, for example. Medical professional 104, or a specialized machine intelligence, can schedule an in-person appointment or remotely adjust patient care through changes in medication or lifestyle. Cardiovascular-function module 214 is also configured to determine trends based on the current hemodynamic characteristic and prior-determined hemodynamic characteristics.

More specifically, cardiovascular-function module 214 is capable of receiving and using sensor data 112, which indicates a patient's skin color, displacement, heart rate, blood pressure, and various other factors. This data may come from single or multiple cardiovascular sensors 106 covering the same or different wavelengths observing multiple locations on the patient's body. With this data, cardiovascular-function module 214 can determine timing relationships, pulse pressure waveforms, and asymmetries in a patient's cardiovascular system. With this data and a circulatory distance between data from different regions of the patient, as well as time synchronizations between the data, cardiovascular-function module 214 can determine a pulse-wave velocity and various simple or highly sophisticated measures of cardiovascular health, including charts of blood pressure, a ballistocardiogram, a photo-plethysmogram (PPG), and pressure-volume loops. Capabilities of cardiovascular-function module 214 are addressed further below.

With regard to cardiovascular sensors 106, three examples of which are shown in FIG. 1, consider a detailed illustration in FIG. 3. Generally, cardiovascular sensors 106 are capable of detecting blood pressure, blood volume, skin color, displacement and so forth at one or more regions of a patient. Cardiovascular sensors 106 may include a radar emitter and receiver, a standard RGB (red, green, blue) camera sensor, a monochrome sensor, a hyperspectral sensor, a stereoscopic sensor, a structured light sensor, a pressure sensor, an ultrasonic sensor, an electrical sensor (e.g., electrocardiograph (ECG) or an impedance cardiograph (ICG)), a reflective or transmissive photoplethysmograph (PPG) sensor, an audio sensor, or combinations of multiple sensors. Example emitters for sensing include one or a combination of nearly any of the electromagnetic spectrum in various forms, such as a combination of sources such as uniform, infrared, tangential, modulated/coded, or coherent (e.g., laser).

Cardiovascular sensors 106 may also have a fixed position or consist of one or more mechanical targeting platforms or those that simply move due to being part of a mobile device. Cardiovascular sensors 106 may also be separated into physically and spatially distinct devices capable of monitoring the body from multiple view angles or observing different regions of the body. Thus, one of cardiovascular sensors 106 may capture an image indicating blood volume at two different regions of patient 102, which then can be compared, by cardiovascular-function module 214, to determine a blood-volume asymmetry or other cardiac function. In the case of a blood-volume asymmetry, a difference in vascular function between the regions may indicate a cardiac-related health problem, such as a stroke. Cardiovascular sensors 106 provide various types of information, and are not limited to determining asymmetries.

In more detail, cardiovascular sensor 106 can be one or a combination of various devices, whether independent, integral with, or separate but in communication with computing device 108. Eight examples are illustrated in FIG. 3, including color and displacement cardiovascular sensor 106-1 (e.g., a camera of computing device 108), sensor 106-2, which is stationary and located within mirror 110, pressure and electrical-sensing mat 106-3, structured-light or stereoscopic sensor system 106-4, optic sensor 106-5 of laptop 108-5, a wearable color and displacement sensor 106-6, which is part of computing spectacles 108-4, radar lamp 106-7, and ultrasonic bathtub 106-8.

Sensor 106-2 is capable of capturing images in an ultraviolet, visible, or infrared optical wavelength. Images recording these wavelengths can be used to determine various changes in blood movement or as calibration signals to detect changes in illumination or patient movement. In some cases blood perfusion and oxygen content can be ascertained, thereby further enabling robust measurement of cardiac function. Due to differential wavelength absorption between human tissue and blood, a hyperspectral sensor can also be used to penetrate the skin to map out veins and arteries to target closer examination for displacement and other measurements.

As noted in part above, pressure and electrical-sensing mat 106-3 is configured to measure the arrival times of cardiac electrical signals (ECG), cardiac generated forces (BCG), and cardiac driven blood flow pulsatility (PPG). The combination of these can sense a pulse-wave velocity of patient 102's blood. This pulse-wave velocity is a measure of a patient's cardiovascular health. The signal-to-noise ratio of the signals from sensor 106-3 can be improved through synchronization with the other sensors to perform correlation techniques such as ensemble averaging and artifact rejection techniques such as motion compensation. The cardiovascular function module 214 can use the time synchronized signals from other sensors to enhance the processing of the signals from sensor 106-3 (e.g., motion activity monitored by sensor 106-2 can be used to compensate and/or selectively weight the signals gathered by sensor 106-3). Alternatively, the time synchronized signals from other sensors can be used to train the system to recognize the patient specific signals generated by cardiovascular events.

Structured-light sensor system 106-4 is capable of projecting structured light at patient 102 and sensing, often with two or more optical sensors, the projected structured light on patient 102 effective to enable capture of images having surface information. This surface information can be used to calculate depth and surface changes for a region of patient 102, such as skin, another organ, or other structure. These changes can be highly accurate, thereby indicating small vibrations and other changes in an organ or structure caused by the cardiovascular system, and thus how that system is operating. Structured-light sensor system 106-4 can, alternatively, be replaced with or supplemented with a targeted, coherent light source for more-accurate displacement measurements. This may include LIDAR (e.g., “light radar” or the process measuring distance by illuminating a target with a laser and analyzing light reflected from the target), laser interferometry, or a process of analyzing light speckle patterns produced by a coherent light on a skin's surface through optical tracking, which enables detection of very small skin displacements.

Radar lamp 106-7 is configured to reflect radiation from human tissue to measure heart rate, respiration rate, and skeletal movement, to name just three examples.

Ultrasonic bathtub 106-8 is configured to generate high-frequency sound waves and to evaluate an echo from those waves. This echo is received at one or more sensors and the time interval between sending and receiving can be measured. These echoes enable analysis of internal body structures. In some cases, acoustic impedance of a two-dimensional cross-section of tissue can be measured, which can measure current heath or a health trend of the measured tissue. Blood flow, tissue movement, blood location, and three-dimensional measurements of structures can also be made. Non-active (no sound waves generated, just receiving sensors) can also be used, though accuracy and robust measurements are more difficult to achieve.

Some of these cardiovascular sensors 106 capture images with sufficient resolution and at sufficient shutter speeds to show detailed colors and displacement, and thus enable determination of mechanical movements or vibrations. These mechanical movements and mechanical vibrations are sufficient to determine a ballistocardiogram (BCG) showing patient 102's cardiac function. Other sensing manners, such as color change or skin displacement in a different region of a patient's body, can be used to establish motion frequency bands to amplify, as well as a timing reference for aggregating multiple heartbeat measurements to improve accuracy of a BCG motion. This BCG information can also be used to provide reference timing information about when a blood pressure pulse leaves the left ventricle and enters the aorta, which combined with the other measurements across the body allow for more-precise estimates of pulse transit times and pulse-wave velocities.

While the BCG signal indicates the timing of the aortic valve, the timing of the atrial valve can be monitored by tracking atrial pressure waveforms visible in the external or internal jugular. This also allows the opportunity to detect atrial fibrillation by detecting missing atrial-pressure pulses. Additionally, aortic-wall stiffness has proven prognostic value in predicting cardiovascular morbidity and mortality. Measuring the pulse-transit time from the start of ejection from the left ventricle into the aorta and up the carotid allows an estimate of that aortic stiffness as well as trending of changes in that stiffness. Thus, determination of arterial-wall stiffness can made independent of blood pressure measurements.

In more detail, cardiovascular sensors 106 are configured to capture sufficient information for the techniques to determine blood asymmetries and other cardiac function, including a pulse-wave velocity of patient 102's blood. This pulse-wave velocity is a measure of a patient's arterial health. In healthy arteries, the pulse-wave velocity is low due to the elasticity of the arteries but, as they harden and narrow, the pulse-wave velocity rises. As blood pressure increases and dilates the arteries, the walls become stiffer, increasing the pulse-wave velocity. While a particular pulse-wave velocity as a snapshot in time may or may not accurately indicate cardiovascular health (e.g., a one-time test at a doctor's office), a change in this pulse-wave velocity (that is, a trend), can be an accurate measure of a change in patient 102's cardiovascular health. If a positive trend, this can reinforce patient 102's healthy habits and, if negative, encourage changes to be made.

Cardiac-related measurements of a patient can include a patient's skin color sufficient to determine a photo-plethysmogram. This PPG measures variations in a size or color of an organ, limb, or other human part from changes in an amount of blood present in or passing through it. These colors and color variations in a patient's skin can show heart rate and efficiency.

These examples show some ways in which the techniques can provide substantially more-valuable (or at least different) data by which to assess a patient's cardiac function than those provided in a medical office or hospital. As noted, conventional health monitoring is often performed at a hospital or medical practitioner's office. Health monitoring at a hospital or office, however, cannot monitor a patient during their normal course of life or as often as desired. This can be a serious limitation because a snapshot captured at a hospital or office may not accurately reflect the patient's health or may not performed at all due to the infrequency of a patient's visits. Even if testing at a hospital or medical office is performed often, it can be inaccurate due to it being of a short duration or due to the testing being in an artificial environment. Note that this does not preclude the use of the techniques disclosed herein at a hospital or medical office, where they may prove valuable in supplementing or replacing conventional measurements, and in the case of in-patient care, may provide a manner for continuous monitoring of patients that are critically (or otherwise) ill.

Returning to FIG. 3, cardiovascular sensor 106 generally may have various computing capabilities, though it may instead be a low-capability device having little or no computing capability. Here cardiovascular sensor 106 includes one or more computer processors 302, computer-readable storage media (CRM) 304, measurement element 306, a wired or wireless transceiver 308 capable of receiving and transmitting information (e.g., to computing device 108), a signal generator 310, and a signal sensor 312.

Measurement element 306 may include various different sensors, from optics, radar, pressure, movement, acceleration, and so forth. Examples includes ultrasonic, pressure, and simple or complex cameras, such as those having low or high shutter speeds, low or high frame rates, low or high resolutions, and having or not having non-visible imaging capabilities.

Signal generator 310 is configured to generate an electromagnetic-spectrum signal, such as the signal received by reception-synchronization module 218 of FIG. 2. Signal generator 310 can be configured to generate electromagnetic-spectrum synchronization signals of various different wavelengths and characteristics, as can signal generator 206 of FIG. 2.

Thus, in the case of multiple cardiovascular sensors 106, each of the cardiovascular sensors 106 may generate a different or otherwise unique electromagnetic-spectrum synchronization signal for reception by reception-synchronization module 218, and then associate the various different electromagnetic-spectrum synchronization signals with respective cardiac sensors 106.

Signal sensor 312 is configured to capture an electromagnetic-spectrum signal, such as the signal generated by signal generator 206 as managed by transmission-synchronization module 220 of FIG. 2.

Computer-readable storage media 304 includes sensor manager 314 and sync-management module 316. Sensor manager 314 is capable of processing sensor data and recording and transmitting sensor data, as well as receiving or assigning appropriate time markers by which to mark or compare the time of various captured images. Sensor manager 314 and cardiovascular-function module 214 may also calibrate measurement element 306 through use of an external sensor. This can aid in calibrating skin colors or displacements to a calibration color or displacement, or even to a cardiac function, such as to a blood pressure or pulse-wave velocity. Thus, assume that one of cardiovascular sensors 106 captures images for two regions while a blood pressure between those regions is also measured through a different device, thereby enabling more-accurate determination of cardiac functions for the cardiovascular sensor and for that patient. Other potential calibration sensors include, but are not limited to, ECG, conventional BCG, digital stethoscopes, ultrasound, and the like. Another example is the use of an external blood pressure meter to calibrate the pulse wave velocity over time to determine long-term changes in arterial-wall stiffness by separating arterial stiffness due to blood pressure versus that due to the dilation by blood pressure.

Sync-management module 316 is configured to generate or receive a signal as noted above, depending on whether reception-synchronization module 218 or transmission-synchronization module 220 is operating at computing device 108. In cases where cardiovascular sensor 106 receives a synchronization signal, marking module 318 can respond with a mark, such as by marking measurements (e.g., an image capture of patient 102's ankle) with an electromagnetic-spectrum synchronization-signal mark associated with the time the signal is received. As noted, this mark enables model 232 to be built.

These and other capabilities, as well as ways in which entities of FIGS. 1-3 act and interact, are set forth in greater detail below. These entities may be further divided, combined, and so on. The environment 100 of FIG. 1 and the detailed illustrations of FIGS. 2 and 3 illustrate some of many possible environments capable of employing the described techniques.

Example Methods

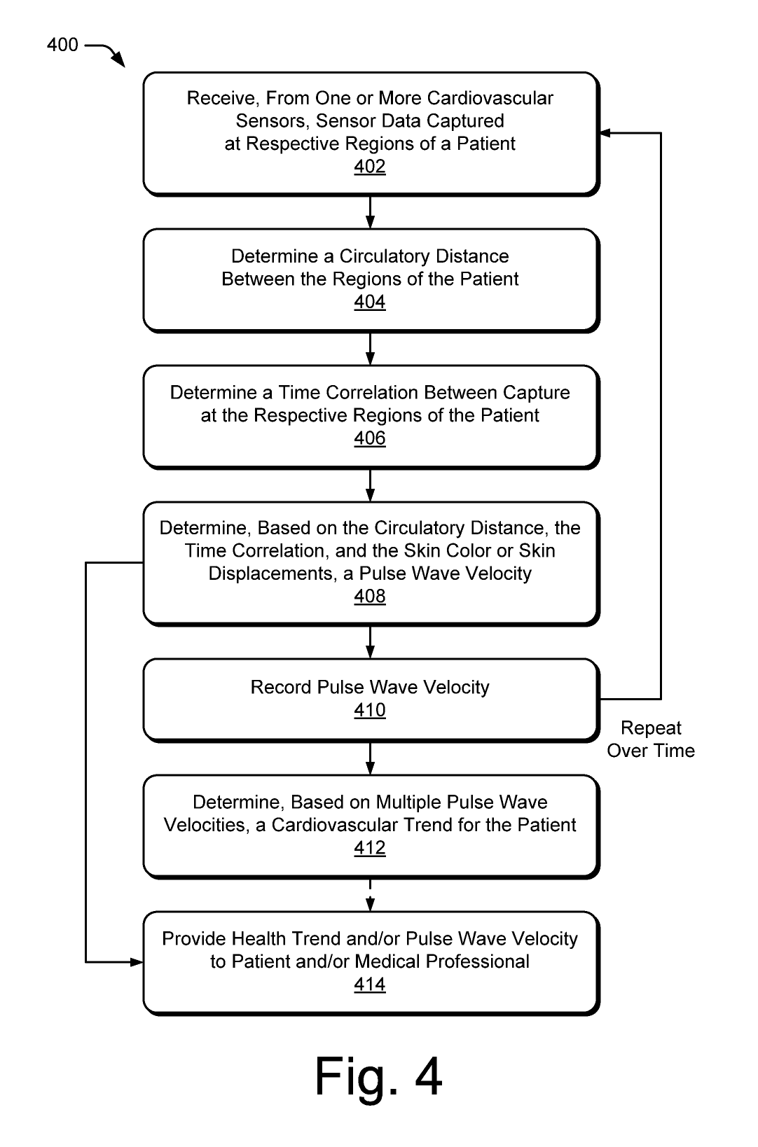

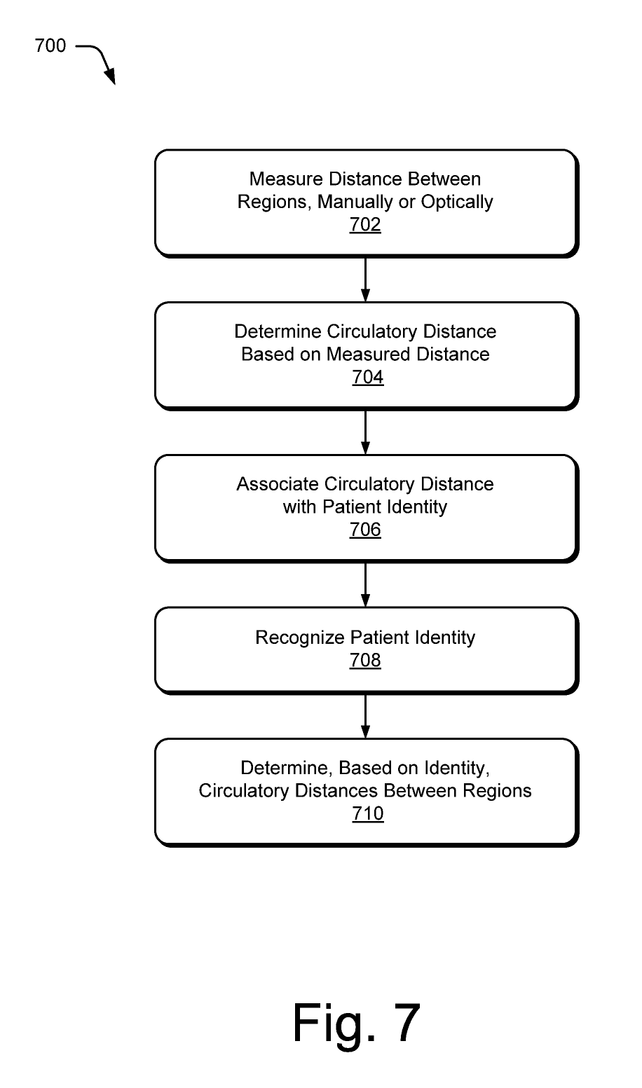

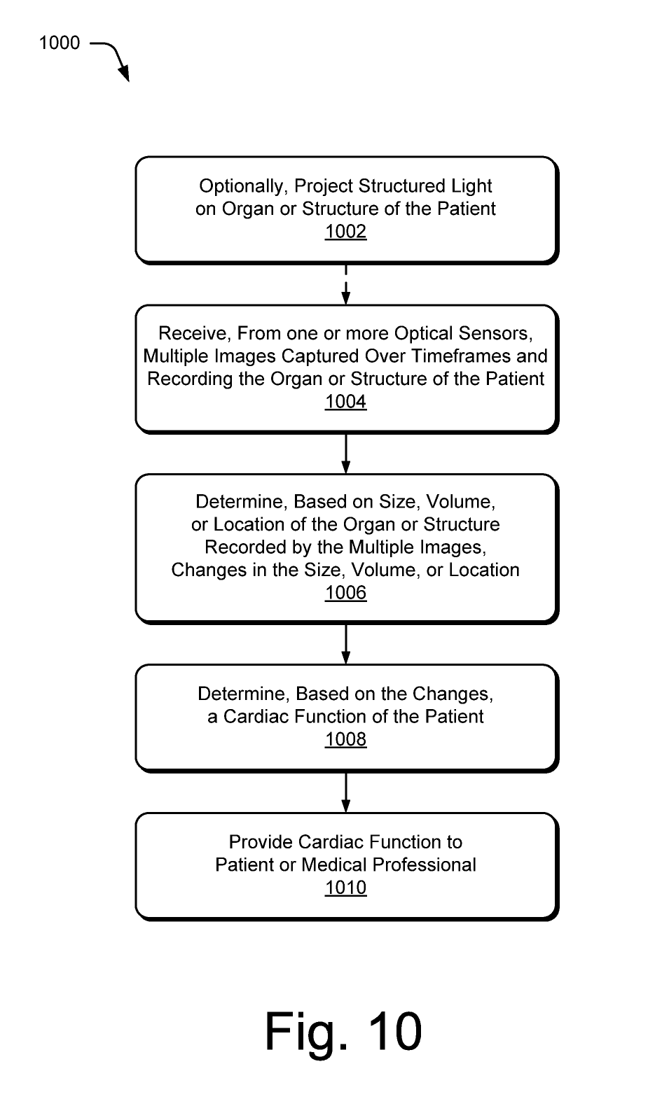

FIGS. 4 and 10 depict methods 400 and 1000, which assess hemodynamic characteristics using a cardiovascular sensor, while FIG. 7 depicts a method 700 for determining a circulatory distance and FIG. 8 depicts a method 800 for synchronizing these cardiovascular sensors. These methods are shown as sets of blocks that specify operations performed but are not necessarily limited to the order or combinations shown for performing the operations by the respective blocks. In portions of the following discussion, reference may be made to environment 100 of FIG. 1 and entities detailed in FIGS. 2 and 3, reference to which is made for example only. The techniques are not limited to performance by one entity or multiple entities operating on one device.

At 402, sensor data is received from one or more cardiovascular sensors. These sensor data are captured at regions of a patient, such as a color captured at a patient's skin on her forehead and a displacement of skin on her neck or on her clavicle. Optionally, as part of operation 402, cardiovascular-function module 214 or sensor manager 314 may automatically determine which regions of a patient are fully visible or partially occluded, and thereby determine better regions of a patient to measure the patient.

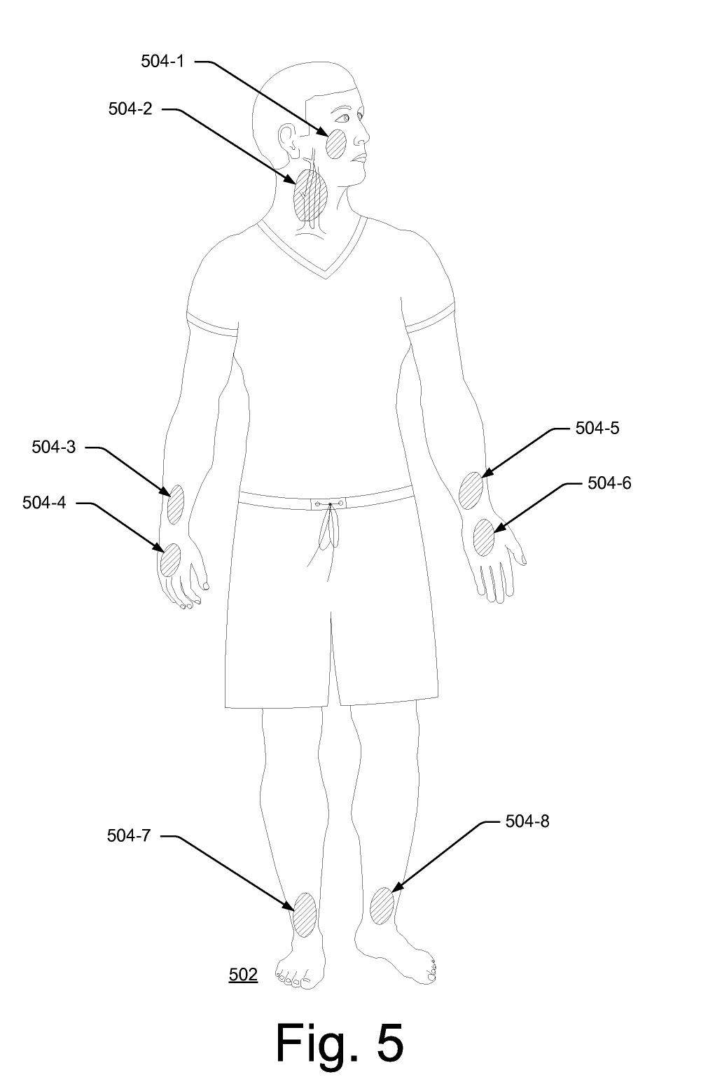

By way of illustration, consider FIG. 5, which shows a male patient 502 having various regions 504 of which images are captured. These regions 504 include, by way example, a cheek region 504-1, a neck region 504-2, an outer wrist region 504-3, an outer hand region 504-4, an inner wrist region 504-5, a palm region 504-6, a front ankle region 504-7, and an inner ankle region 504-8, to name but a few. By way of an ongoing example, assume that one cardiovascular sensor captures a displacement of skin at neck region 504-2 and another a color change of skin at inner wrist region 504-5.

At 404, a circulatory distance is determined between the regions of the patient at which the colors or displacements are captured. This circulatory distance can be an approximation based on a linear distance between the regions, such as a linear distance based on an axial distance oriented relative to an axis of the patient's spine, or simply a vertical distance with the patient standing. In some cases, however, the techniques determine or approximate a circulatory distance based on an arterial-path distance. This arterial-path distance can be determined or approximated using an arterial structure of the patient or determined based on a skeletal structure of the patient, including automatically by optical visualization of the patient.

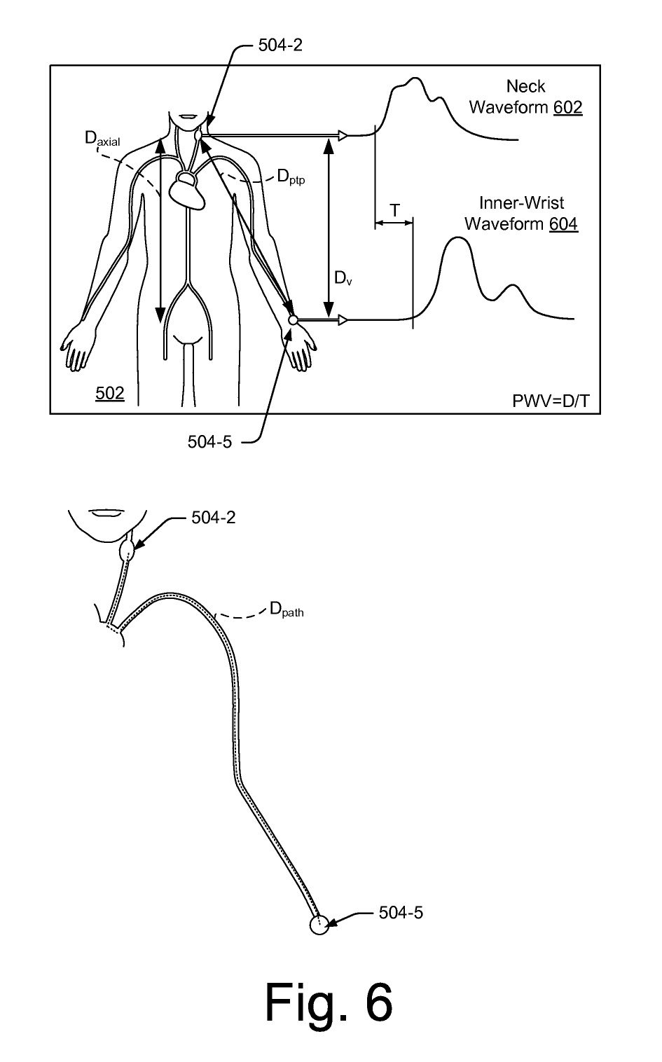

By way of illustration of the various circulatory distances that can be used, consider FIG. 6. Here assume that multiple images are captured of patient 502's neck region 504-2 (also shown in FIG. 5) sufficient to determine a neck waveform 602. Multiple images are also captured of inner wrist region 504-5 sufficient to determine an inner-wrist waveform 604. At operation 404, cardiovascular-function module 214 determines the circulatory distance from neck region 504-2 and inner wrist region 504-5 in one of the following four manners. In the first, a vertical distance Dᵥ is calculated with the patient standing. In the second, an axial distance Daxiai is calculated based on the distance relative to an axis of the patient's spine—here it is similar to the vertical distance, Dᵥ, but if the person is oriented at an angle, the distances are different. In the third, cardiovascular-function module 214 calculates the distance as a point-to-point between the regions, here shown as Dₚₜₚ. In the fourth, cardiovascular-function module 214 calculates or approximates the distance that blood travels through patient 502's arteries, Dₚₐₜₕ. This arterial-path distance can be determined based on the arteries themselves or an approximation based on a skeletal structure or an overall body shape of the person. Data for skeletal structure and overall body shape can be determined using images captured for the regions and structures in between the regions, optically or otherwise. In some cases, radar can be used that penetrates clothing to track bony surfaces, thereby providing a skeletal structure from which arterial distance can be approximated.

While not required, operation 404 may be performed, in whole or in part, using method 700 illustrated in FIG. 7, which is described below. By way of overview, in this example method, the techniques determine one or more of the distances illustrated in FIG. 6.

The more-accurate distance calculations provide a better pulse-wave velocity, and thus indicate a current hemodynamic characteristic. While potentially valuable, more-accurate distances are not necessarily required to show trends in hemodynamic characteristics. Trends are provided by consistently calculated distances more than accurate distances, and for a specific individual, should not change significantly over time for the same measurement points. If the measurement points vary due to visibility issues (such as clothing), then distance measurement estimates increase in importance for accurate trending.

At 406, a time correlation between capture of the sensor data is determined. This time correlation can be performed by timing module 216, reception-synchronization module 218, or transmission-synchronization module 220 as noted above. While not required, operation 406 may be performed, in whole or in part, using method 800 illustrated in FIG. 8, which is described below. By way of overview, in this example method, the techniques determine one or more of the time correlations illustrated in FIG. 6 as well as time synchronizations as illustrated in FIG. 9. Time correlations address times between same or similar cardiac events. Thus, a time “T” of FIG. 6 shows a time correlation between a start of a same heartbeat at two different regions of patient 502. This time “T” is used to determine a pulse-wave velocity, for example. In short, time correlations correlate multiple measurements of a same event, such as a peak-to-peak heart rate at two regions. Time synchronizations, however, precisely assign times at which the measurements are made. Thus, time synchronization permit accurate time correlations. For example, if inner-wrist waveform 604 is measured 1/100^{th} of one second after neck waveform 602 but both are received for analysis at a same time; a time correlation between the two measurements would be inaccurate by 1/100^{th} of a second but for the time synchronization. Moreover, this inaccuracy would therefore indicate an inaccurate PWV.

In more detail, cardiovascular-function module 214 may determine correlations between sensor data based on a time at which a maximum or minimum blood volume is determined for each of the regions, or some other consistent and comparable point in a waveform, such as a beginning of a pressure increase in the waveform (show in FIG. 6). This time correlation can be considered a temporal distance between multiple images capturing some measure of cardiac operation, such as blood volume at each of the regions. Thus, by comparing various images or other sensor data for regions, cardiovascular-function module 214 can correlate sensor for a same heartbeat or other hemodynamic characteristic.

Note that waveforms 602 and 604 can be determined through color, or in some locations of the body, related waveforms can be determined through displacement. Cardiovascular-function module 214 can determine, based on a change in color to regions over time, a waveform. These color changes indicate a peak or crest of a wave based on blood content at the organ and thus can be used to determine a shape of the wave. While a shape of a wave can differ at different regions, they can still be compared to find a time correlation. In the case of lower-than-desired optical frame rates due to sensitivity or processing limitations, interpolation or curve fitting can be used to improve the estimate of the waveform for improved time correlation. Repeated measurements, which are time shifted relative to the pressure wave either naturally by the optical sampling frequency or intentionally by adjusting the sampling frequency, can build up a super-sampled estimate of the waveform. The higher timing-resolution waveform can be used for more-accurate timing measurements. Additionally, displacements, through either direct distance measurements or tangential shading, can show signals related to the pressure waveforms as the arteries and veins expand and contract. These waveforms can further reveal cardiac activity, such as valve timing, valve leakage (regurgitation), fibrillation, stroke volume, and the like.

At 408, a pulse-wave velocity for blood circulation through the patient is determined based on the circulatory distance and the time synchronization, as well as the skin colors or displacements. As shown in FIG. 6, the time synchronization is based on similar points in a waveform and the circulatory distance is some calculation or approximation of the distance blood travels from regions at which images are captured. In more detail, a pulse-wave velocity is the circulatory distance divided by the time synchronization.

Pulse-wave velocity is a good measure of cardiac function. It can indicate, for example, an arterial stiffness of a patient (the faster the pulse-wave velocity, the higher the arterial stiffness), a blood pressure, and a mean arterial pressure for the patient. In more detail, the techniques can determine blood pressure based on the pulse-wave velocity using the Bramwell-Hill equation, which links pulse-wave velocity to compliance, blood mass density, and diastolic volume. Each of these are measures of cardiac function that can indicate a patient's cardiac health. As noted above, the techniques can provide these cardiac functions to a patient, thereby encouraging the patient to make changes or, in some cases, seek immediate medical care.

Note that, in some cases, three or more different regions are measured at operation 402. In these cases, cardiovascular-function module 214 may determine which of the regions are superior to others, such as due to data captured for those regions being noisy or incomplete or otherwise of inferior quality. Those that are superior can be used and the others discarded, or cardiovascular-function module 214 may weigh the determined pulse wave velocity between different regions based on the quality of the data used to determine those pulse wave velocities. This can be performed prior to or after recording those pulse wave velocities as described below.

Following determination of the pulse-wave velocity at operation 408, the techniques may proceed to record the pulse-wave velocity at operation 410 and then repeat operations 402-410 sufficient to determine a trend at operation 412. In some cases, however, the determined pulse-wave velocity is provided, at operation 414, to the patient or medical professional. Optionally, calibration data from an external sensor can be used to improve performance. For example, an external blood pressure monitor could be used to train the system to correlate PWV with blood pressure. The device could be captured through an electronic network (BluetoothTM or the like) or the optical system could scan the user interface and perform OCR to read the results. Machine learning could be applied to create a patient-specific model for estimating blood pressure from PWV.

At 412, a cardiovascular trend for the patient is determined based on multiple pulse-wave velocity measurements, such as comparing prior and later-time determined pulse-wave velocities. This can simply show a trend of pulse-wave velocities rising or falling, such as with velocity rising due to increased arterial stiffness. Multiple locations across the body can be measured to map changes over time. Cardiovascular-function module 214 may also determine other measures of cardiac function, such as changes in flow asymmetries or pulse pressure waveforms over time.

At 414, as noted, this trend determined at operation 412, or a pulse-wave velocity determined at operation 408, is provided to the patient or a medical professionals, e.g., patient 102 or 502 and medical professional 104, of FIG. 1 or 6.

In some cases skin color, skin displacement, or both are used by the techniques in method 400. Thus, color changes can indicate blood flow over time, as can displacement changes. Furthermore, use of color and displacement both can indicate an amount of blood in capillaries in the skin while displacement can indicate a change to a volume of the skin or an organ under the skin, such as vein or artery, and thus an amount of blood in the skin or near it can be determined.

Note also that the techniques may repeat operations of method 400 for various other regions. Doing so may aid in altering the pulse-wave velocity to improve its accuracy or robustness by determining another pulse-wave velocity between two other regions or between another region and one of the regions for which images are captured. Thus, the techniques may determine a pulse-wave velocity for the patient based on two pulse-wave velocities between regions, such as regions 504-3 and 504-1, 504-7 and 504-1, and/or 504-8 and 504-2.

As noted above, method 400 can be supplemented, and operation 404 may be performed, in whole or in part, using method 700 illustrated in FIG. 7. In this example method, the techniques determine one or more of the distances illustrated in FIG. 6. For operations 702-706, a patient's circulatory distances between regions are establish for later use as a manner in which to calibrate the patient's distances. While calibration for a single sensing milieu to determined trends may not be required, use of different sensing milieus or to determine a hemodynamic characteristic with quantitative precision both aid from use of calibration. Operation 708 and 710 can be used as one way in which the techniques may perform operation 404 of method 400.

At 702, a distance between various regions is measured, optically, manually, or in other manners. Consider, for example, capturing an image of patient 502 of FIG. 5. Assume that some physical data is available, such as a distance between the cardiovascular sensor capturing the image and patient 502, or a height of patient 502, and so forth. With this physical data, the distance can be determined from the image. Generally, this distance is from point-to-point, and is later analyzed for circulatory distance. Other manners can also or instead be used, such as a nurse measuring patient 502, either from point-to-point or along structures, such as from a wrist to an elbow, elbow to shoulder, and from shoulder to heart. A patient may also interact with cardiovascular sensor 106 and cardiovascular-function module 214 to calibrate distances between regions, such as standing at a particular location relative to cardiovascular sensor 106 and so forth. Various other technologies can be used as well, such as structured light cardiovascular sensors, radar, LIDAR, and SODAR (measuring distance through use of sound through air).

At 704, a circulatory distance is determined using the measured distance. In some cases the measured distance is simply used as the circulatory distance, such as measuring Dₚₜₚ and then using Dₚₜₚ (of FIG. 6) as the circulatory distance. As noted in part herein, however, other circulatory distances may be determined, such as measuring a point-to-point where patient 502's arm is bent, and thus calculating a fully extended point-to-point to maintain consistency of circulatory distance. Other examples include measuring Dᵥ and then, based on data about patient 502, determining an arterial-path distance (Dₚath).

At 706, these various determined circulatory distances are associated with the patient's identity. The identity of the patient can be entered, queried from the patient, or simply associated with some repeatable measure of identity, even if the person's name is not known. Examples include determining identity using fingerprints or facial recognition, and then associating distances with that fingerprint or facial structure.

At 708, the patient's identity is determined. This can be performed as part of operation 404. With this identity, at 710 circulatory distances between regions are determined. For example, cardiovascular-function module 214 may use facial recognition to identify patient 502 and, after determining patient 502's identity, find previously determined cardiovascular distances between each of regions 504 by simply mapping the relevant regions to previously stored distances. When cardiovascular time synchronizations are determined at operation 406, a pulse wave velocity can be determined using the mapped-to cardiovascular distance for the regions measured.

FIG. 8 depicts a method 800 for synchronizing cardiovascular sensors for cardiovascular monitoring. Method 800 may work alone or in conjunction with other methods or operations thereof, such as to improve a time correlation of operation 406 of method 400.

At 802, an electromagnetic-spectrum synchronization signal is transmitted. This can be performed by electromagnetic-spectrum signal generator 206 of FIG. 2. Example electromagnetic-spectrum signals include those within the visual range, infrared range, radio-frequency range to name just a few. Operation 802 may transmit multiple or different kinds of electromagnetic-spectrum synchronization signals though this is not required.

By way of illustration, consider an example shown in FIG. 9. FIG. 9 illustrates sensing milieu 900, which includes sensors 106-1, 106-2, 106-3, and 106-6. Sensing milieu 900 also includes patient 102 and computing spectacles 108-4. In this example, transmission synchronization module 220 causes signal generator 206, both of the computing spectacles 108-4, to transmit one or more electromagnetic-spectrum synchronization signals. This transmission is shown with arrows from the generator to the sensors 106. Computing spectacles 108-4 is here acting as both a computing device and a cardiovascular sensor, wearable color and displacement sensor 106-6, while sensor 106-1, which is part of smartphone 108-1, is acting only as a sensor.

Here the illustration assumes that sensors 106 receive the electromagnetic-spectrum synchronization signal and use it as a timing marker of some sort. Thus, each sensor may use the electromagnetic-spectrum synchronization signal to mark cardiovascular measurements made by each of the cardiovascular sensors. Sensor 106-3, for example, may mark cardiovascular measurements at the instant the electromagnetic-spectrum synchronization signal is received, or record the time received and use it in a response by which the techniques may determine times synchronizations between the various sensors 106.

At 804, a response having an electromagnetic-spectrum synchronization signal mark is received from each of the cardiovascular sensors. This response can simply be cardiovascular measurements having the electromagnetic-spectrum synchronization signal mark. In some other cases, response includes a timing indicator that can be associated with a later-received cardiovascular measurement.

However received, the electromagnetic-spectrum synchronization-signal marks for the cardiovascular sensors can be used to time synchronize the cardiovascular measurements received by the computing device. In some cases, the time synchronization corrects for time differences in processing reception of the electromagnetic-spectrum synchronization signal and transmitting the response. In cases where the mark is included with the cardiovascular measurements, the time synchronization corrects for processing, transmission (e.g., differences in wired or wireless transmission protocols), and other timing effects. These timing effects can be relatively permanent or vary due to a position of patient 102 or 502, or changes in processing or transmission speeds. Because of this, the techniques may select to resynchronize the cardiovascular sensors regularly, even as often as every heartbeat.

As part of this method, the signal generation time at which an electromagnetic-spectrum synchronization signal is transmitted can be precisely assigned by timing module 216 of FIG. 2, shown at reception times 230.

Continuing the ongoing example of FIG. 9, computing spectacles 108-4 receive responses from each of sensors 106-1, when 106-2, and 106-3. Note that sensor 106-6 is included, or integral with, computing spectacles 108-4, and thus may or may not receive and respond to the electromagnetic-spectrum synchronization signal. In some cases, even an integral sensor can benefit from receiving and responding to the electromagnetic-spectrum synchronization signal as it permits the techniques to determine processing delays within the computing device. Reception of the responses is shown from the sensors with the arrows pointing towards computing spectacles 108-4.

At 806, a time synchronization is determined between two or more cardiovascular sensors based on the received marks. Continuing the ongoing example, consider timing chart 902. In timing chart 902, the electromagnetic-spectrum synchronization signal is transmitted at time So. Assume that based on processing transmission and other timing effects, that sensor 106-1 marks reception of the electromagnetic-spectrum synchronization signal and then transmits the cardiovascular measurements with the marking, shown received by computing spectacles 108-4 at Mi. Therefore, the time synchronization between the signal being transmitted and the cardiovascular measurements being received is Tsi. Similarly, for sensor 106-2, cardiovascular measurements are received with M2 for a time synchronization of Tse. Likewise, for sensors 106-3 and 106-6, cardiovascular measurements are received with M3 for a time synchronization of Ts3 and M6 for a time synchronization of Ts6, respectively. At this point, the cardiovascular measurements can be synced together relative to any one of the markings, such as M6, or the signal transmission time of So. Note that these synchronization times are shown longer than is commonly the case to better illustrate the effect. Each of these time synchronizations enables better time correlations, such as those shown in FIG. 6.

The cardiovascular measurements shown in timing chart 902 include three waveforms and a displacement ballistocardiogram. Waveform 904 is determined based on measured color and displacement sensor 106-1 recording color changes at patient 102's lower left ankle (see region 504-8 in FIG. 5). Waveform 906 is determined based on hyperspectral sensor 106-2 recording displacement changes at patient 102's neck (see region 504-2 in FIG. 5). Waveform 908 is a displacement ballistocardiogram determined based on pressure readings received through pressure and electrical-sensing mat 106-3. Waveform 910 is determined based on wearable color and displacement sensor 106-6 recording color and displacement changes at patient 102's right wrist (see region 504-3 at FIG. 5). Note that the time synchronization for sensor 106-6 is the shortest of the four time synchronizations due to sensor 106-6 being internal to, and thus using wired transmission, within computing spectacles 108-4, although in many cases internal processing delays exceed any transmission time delays. As illustrated in FIG. 2, transmission-synchronization module 220 determines time synchronizations 234 for each of sensors 106-1, 106-2, 106-3, and 106-6.

At 808, time synchronizations are provided effective to enable cardiovascular measurements to be synchronized to determine hemodynamic characteristics of the patient. Continuing the ongoing example of FIG. 9, transmission-synchronization module 220 provides time synchronizations 234 to reception-synchronization module 218.

At 810, a model is determined based on the synchronization times for each of the sensors. Concluding the ongoing example, reception-synchronization module 218 determines model 232 for the sensing milieu shown in FIG. 9. This model is effective to enable cardiovascular measurements by the cardiovascular sensors to be synchronized.

Note that operations of method 800 can be performed multiple times, for a single synchronization or for multiple synchronizations performed periodically. Thus, electromagnetic-spectrum signal generator 206 may transmit different signals (e.g., signals having different wavelengths) for different sensors, such as signals having different wavelengths or other different characteristics. Assume, for illustration, that the example shown in FIG. 9 is modified such that an infrared electromagnetic-spectrum synchronization signal is sent, received, in response is received back from sensor 106-2. This can be based on the synchronization capabilities of the particular sensors and, even if the different synchronization signals are generated at different times, the techniques can determine which of the signals was received by the particular sensor. Here sensor 106-3 is known to be configured to receive radio band synchronization signals but not visible light signals. Also, sensor 106-1 is configured to receive a signal in the visible range, as it is integral with smartphone 108-1. Sensor 106-6 is configured in this example to receive signals in the visible range as well, even though alternatively it could receive a signal internally, as that calibrates out potential variable processing delays in the receive path. Markings received from each of the sensors therefore can be correlated to the particular generation time of the particular signal received by the sensor.

FIG. 10 depicts a method for assessing hemodynamic characteristic using optical sensors and based on size, volume, or location of an organ or structure of a patient sensed through those optical sensors. In method 1000, images are captured over 2 to 10 millisecond-range or faster timeframes, thereby providing multiple images relating to an organ or structure of the patient. Note that sub-millisecond timeframes can also be useful for measure acoustic vibrations and are optional. Method 1000 may operate, in whole or in part, in conjunction with method 400, though this is not required.

At 1002, structured light is projected onto an organ or structure of a patient. Note that this is optional, though in some cases use of structured light aids in accurate measurement of movement and displacement of a region of the patient. Alternatively, tangential light may be used to generate shadowing to detect skin displacement, or a coded light source could be used to reject external interference. For example, an alternating on and off light source at the frame rate would allow sampling and canceling of the background illumination. Further, light reflected from background objects or patient clothing can be used to track changes in lighting over time or in different conditions, e.g., daylight vs night, light bulb luminosity degradation over time, and so forth. With this data, ambient light and its effect on images captured can be calibrated and for which cardiovascular-function module 214 can adjust for the various methods described herein.

At 1004, multiple images are received that capture an organ or structure of a patient. As noted, the images captured may include capture of structured light to aid in determining displacement using surface information captured. This surface information can be from one or multiple devices. These multiple images can be received from one or multiple cardiovascular sensors and over various timeframes, such as those captured at millisecond-range or faster timeframes.

At 1006, changes in the size, volume, or location of the organ or structure of the patient are determined. These changes are determined by comparing sizes, volumes, or locations of the organ or structure of the patient recorded by the various multiple images captured over time. Note that these changes can be used in coordination with, or to compensate for, data from methods 400, and vice-versa. Thus, data from one portion of the body captured in any of the various manners described herein can be used to compensate for other data, such as using a color or waveform determined at method 400 to compensate for motion artifacts in the data of method 1000.

At 1008, a cardiac function of the patient is determined based on the changes. This cardiac function can be one of the many described above, including heart rate, blood pressure, pulse-wave velocity, pressure volume loops, blood-volume and other asymmetries, and so forth, as well as respiration rate.

By way of a first example, consider a case where an asymmetry is determined between to different regions of the patient. In some cases this asymmetry is determined by blood-volume differences, which can be indicated by size or color. To determine an asymmetry, cardiovascular-function module 214 may compare the different cardiovascular pulse times of the regions, where one of the pulse times for a same heartbeat is different, as it is further from the patient's heart. Alternatively, the waveform's peak, median, or trough of blood volume can be accurately compared. Thus, assume that a right wrist and a left wrist of a patient have different blood volumes at each of their peaks, with one being a lower peak blood volume that the other, thereby indicating some difference in vascular function.

Cardiac function trends, as noted in part above, can greatly aid in helping patients maintain or change their habits to improve their cardiac health. Consider, for example, a trend showing a change to a hemodynamic characteristic over weeks, months, or years using the techniques. This trend can show cardiac function in many ways superior to the best invasive cardiac testing because a trend need not require perfect accuracy—instead consistency is used. Furthermore, this can be performed by the techniques without interrupting the patient's day, making the patient perform a test, or requiring the patient to go see a medical professional. By so doing, many lives can be saved.

In more detail, consider the techniques in the context of FIGS. 1-3. Here various kinds of cardiovascular sensors 106 sense regions (e.g., regions 504 of FIG. 5) of a patient (e.g., patient 102 of FIG. 1 or patient 502 of FIGS. 5 and 6) through measurement elements 306. This sensor data (e.g., images) are then processed and/or stored by sensor manager 314 (e.g., to mark the images with times), after which they are passed, through wired/wireless transceiver 308 as sensor data 112 to cardiovascular-function module 214 operating on computing device 108 of FIG. 2. Also passed are indications of the region and dates 222 at which the sensor data 112 was captured.

Cardiovascular-function module 214 then performs operations of method 400, 700, 800, and/or method 1000 to determine cardiac function, as noted above. Consider, for example, a case where cardiovascular-function module 214 determines that a cardiac function meets or exceeds a safety threshold. Example safety thresholds include a blood pressure being too high, a heart rate being too rapid or irregular, or a low blood-oxygen level. This safety threshold can also be complicated or more difficult to determine, such as a patient's heart showing an end-diastolic volume ejected out of a ventricle during a contraction being less than 0.55 (this is a measure of ejection fraction (EF) and low fractions can indicate a heart attack is imminent). These are but a few of the many safety thresholds for cardiac function enabled by the techniques. If a safety threshold is exceeded, medical professional 104 (or family/caretaker) and patient 102 can be informed, such by operation 1010 of method 1000.

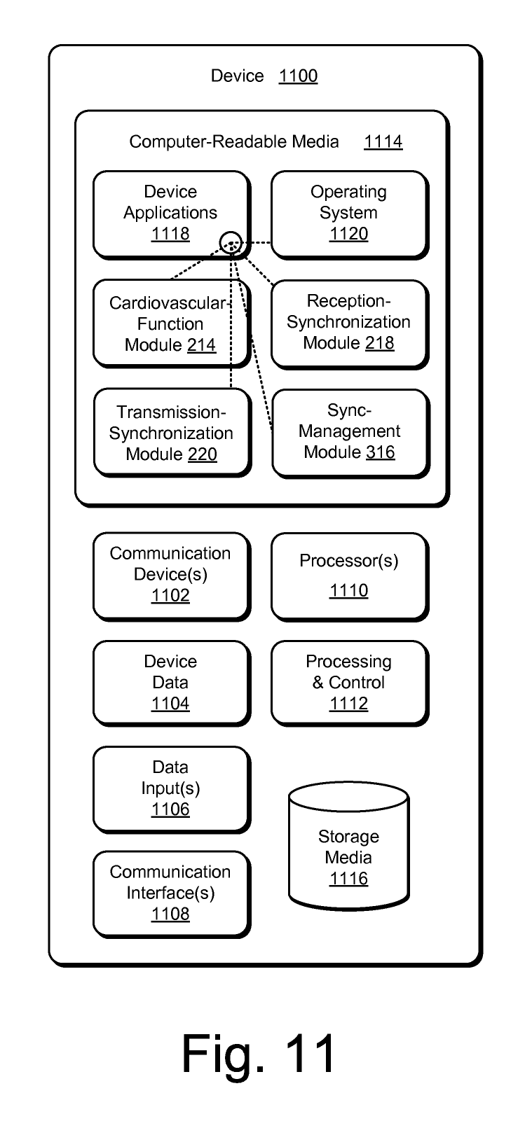

The preceding discussion describes methods relating to assessing cardiac function and synchronizing cardiovascular sensors for cardiovascular monitoring for a human cardiovascular system. Aspects of these methods may be implemented in hardware (e.g., fixed logic circuitry), firmware, software, manual processing, or any combination thereof. These techniques may be embodied on one or more of the entities shown in FIGS. 1-3, 9, and 11 (computing system 1100 is described in FIG. 11 below), which may be further divided, combined, and so on. Thus, these figures illustrate some of the many possible systems or apparatuses capable of employing the described techniques. The entities of these figures generally represent software, firmware, hardware, whole devices or networks, or a combination thereof.

Example Computing System

FIG. 11 illustrates various components of example computing system 1100 that can be implemented as any type of client, server, and/or computing device as described with reference to the previous FIGS. 1-10. In embodiments, computing system 1100 can be implemented as one or a combination of a wired and/or wireless wearable device, System-on-Chip (SoC), and/or as another type of device or portion thereof. Computing system 1100 may also be associated with a user (e.g., a patient) and/or an entity that operates the device such that a device describes logical devices that include users, software, firmware, and/or a combination of devices.

Computing system 1100 includes communication devices 1102 that enable wired and/or wireless communication of device data 1104 (e.g., received data, data that is being received, data scheduled for broadcast, data packets of the data, etc.). Device data 1104 or other device content can include configuration settings of the device, media content stored on the device, and/or information associated with a user of the device. Media content stored on computing system 1100 can include any type of audio, video, and/or image data, including complex or detailed results of cardiac function determination. Computing system 1100 includes one or more data inputs 1106 via which any type of data, media content, and/or inputs can be received, such as human utterances, user-selectable inputs (explicit or implicit), messages, music, television media content, recorded video content, and any other type of audio, video, and/or image data received from any content and/or data source.

Computing system 1100 also includes communication interfaces 1108, which can be implemented as any one or more of a serial and/or parallel interface, a wireless interface, any type of network interface, a modem, and as any other type of communication interface. Communication interfaces 1108 provide a connection and/or communication links between computing system 1100 and a communication network by which other electronic, computing, and communication devices communicate data with computing system 1100.

Computing system 1100 includes one or more processors 1110 (e.g., any of microprocessors, controllers, and the like), which process various computer-executable instructions to control the operation of computing system 1100 and to enable techniques for, or in which can be embodied, such as synchronizing cardiovascular sensors for cardiovascular monitoring. Alternatively or in addition, computing system 1100 can be implemented with any one or combination of hardware, firmware, or fixed logic circuitry that is implemented in connection with processing and control circuits, which are generally identified at 1112. Although not shown, computing system 1100 can include a system bus or data transfer system that couples the various components within the device. A system bus can include any one or combination of different bus structures, such as a memory bus or memory controller, a peripheral bus, a universal serial bus, and/or a processor or local bus that utilizes any of a variety of bus architectures.

Computing system 1100 also includes computer-readable media 1114, such as one or more memory devices that enable persistent and/or non-transitory data storage (i.e., in contrast to mere signal transmission), examples of which include random access memory (RAM), non-volatile memory (e.g., any one or more of a read-only memory (ROM), flash memory, EPROM, EEPROM, etc.), and a disk storage device. A disk storage device may be implemented as any type of magnetic or optical storage device, such as a hard disk drive, a recordable and/or rewriteable compact disc (CD), any type of a digital versatile disc (DVD), and the like. Computing system 1100 can also include a mass storage media device 1116.

Computer-readable media 1114 provides data storage mechanisms to store device data 1104, as well as various device applications 1118 and any other types of information and/or data related to operational aspects of computing system 1100. For example, an operating system 1120 can be maintained as a computer application with computer-readable media 1114 and executed on processors 1110. Device applications 1118 may include a device manager, such as any form of a control application, software application, signal-processing and control module, code that is native to a particular device, a hardware abstraction layer for a particular device, and so on.

Device applications 1118 also include any system components, modules, or managers to implement the techniques. In this example, device applications 1118 include cardiovascular-function module 214, reception-synchronization module 218, transmission-synchronization module 220, or sync-management module 316.

Conclusion

Although embodiments of techniques for, and apparatuses enabling, synchronizing cardiovascular sensors for cardiovascular monitoring have been described in language specific to features and/or methods, it is to be understood that the subject of the appended claims is not necessarily limited to the specific features or methods described. Rather, the specific features and methods are disclosed as example implementations of these techniques.

What is claimed is:

1. A system comprising:
one or more computer processors;
an electromagnetic-spectrum sensor, the electromagnetic-spectrum sensor configured to capture electromagnetic-spectrum synchronization signals from cardiovascular sensors oriented to a patient; and
one or more computer-readable media having instructions stored thereon that, responsive to execution by the one or more computer processors, enables modules comprising:
a timing module, the timing module configured to precisely assign a reception time to each of the captured electromagnetic-spectrum synchronization signals from the cardiovascular sensors; and
a reception-synchronization module configured to model, based on the precisely assigned reception times for the captured electromagnetic-spectrum synchronization signals, cardiovascular timing of the cardiovascular sensors effective to enable cardiovascular measurements from the cardiovascular sensors to determine a hemodynamic characteristic of the patient.

2. The system of claim 1, wherein the reception-synchronization module is further configured to:
receive an electromagnetic-spectrum synchronization signal generation mark and a cardiovascular measurement from each of the cardiovascular sensors; and
synchronize the cardiovascular measurements using the precisely assigned reception times and the electromagnetic-spectrum synchronization signal generation marks.

3. The system of claim 1, wherein the captured electromagnetic-spectrum synchronization signals from the cardiovascular sensors have different wavelengths and the reception-synchronization module is further configured to assign each of the captured electromagnetic-spectrum synchronization signals to respective of the cardiovascular sensors based on the different wavelengths.

4. The system of claim 3, wherein the different wavelengths are previously associated with each of the cardiovascular sensors and the reception-synchronization module is further configured to assign the captured electromagnetic-spectrum synchronization signals to respective of the cardiovascular sensors based on the different wavelengths and the previous association.

5. The system of claim 1, wherein the electromagnetic-spectrum synchronization signal is in an optical, radio-frequency, or infrared bandwidth.

6. The system of claim 1, wherein the captured electromagnetic-spectrum synchronization signals from the cardiovascular sensors have different pulse characteristics or contain data and the reception-synchronization module is further configured to assign each of the captured electromagnetic-spectrum synchronization signals to respective of the cardiovascular sensors based on the different pulse characteristics or the contained data.

7. The system of claim 6, wherein the different pulse characteristics are previously associated with each of the cardiovascular sensors and the reception-synchronization module is further configured to assign the captured electromagnetic-spectrum synchronization signals to respective of the cardiovascular sensors based on the different pulse characteristics.

8. The system of claim 1, wherein the reception-synchronization module is further configured to identify and store the captured electromagnetic-spectrum synchronization signals with the precisely assigned reception times of the respective cardiovascular sensors.

9. The system of claim 1, wherein the hemodynamic characteristic includes pressure waves representing blood flow through an artery or vein of the patient.

10. A computer-implemented method comprising:
transmitting an electromagnetic-spectrum synchronization signal;
receiving responses from respective cardiovascular sensors, the responses having electromagnetic-spectrum synchronization signal marks;
determining time synchronization between the respective cardiovascular sensors based on the electromagnetic-spectrum synchronization signal marks; and
providing the time synchronizations effective to enable determination of a model by which cardiovascular measurements received from each of the respective cardiovascular sensors can be synchronized.

11. The computer-implemented method as described in claim 10, wherein the electromagnetic-spectrum synchronization signal is in an optical, radio-frequency, or infrared bandwidth.

12. The computer-implemented method as described in claim 10, further comprising determining the model.

13. The computer-implemented method as described in claim 10, further comprising:
determining a circulatory distance between regions of a patient being measured by each of the respective cardiovascular sensors and to which each of the cardiovascular measurements are associated;
determining a time correlation between capture of the cardiovascular measurements at the respective regions of the patient;
determining, based on the circulatory distance, the time correlation, and the time synchronizations of the model, a hemodynamic characteristic of the patient.

14. The computer-implemented method as described claim 13, wherein the hemodynamic characteristic is a pulse-wave velocity.

15. A system comprising:
one or more computer processors;
an electromagnetic-spectrum signal generator, the electromagnetic-spectrum signal generator configured to send an electromagnetic-spectrum synchronization signal capable of capture by two or more cardiovascular sensors; and
one or more computer-readable media having instructions stored thereon that, responsive to execution by the one or more computer processors, enable a transmission-synchronization module configured to:
receive, from each of the cardiovascular sensors, a response having an electromagnetic-spectrum synchronization-signal mark;
determine a time synchronization between the cardiovascular sensors based on the electromagnetic-spectrum synchronization-signal mark received from each respective cardiovascular sensor; and
provide the time synchronization effective to enable cardiovascular measurements by the cardiovascular sensors to be synchronized to determine a hemodynamic characteristic of a patient.

16. The system of claim 15, wherein the electromagnetic-spectrum synchronization-signal marks for two of the cardiovascular sensors correct, for a single electromagnetic-spectrum synchronization signal received by both of the two cardiovascular sensors, time differences in processing reception of the electromagnetic-spectrum synchronization signal and transmitting the response.

17. The system of claim 15, wherein the electromagnetic-spectrum synchronization-signal mark is included within the cardiovascular measurement by the one of the cardiovascular sensors.

18. The system of claim 15, wherein the instructions, responsive to execution by the one or more computer processors, further enables a timing module, the timing module configured to precisely assign a signal generation time to the electromagnetic-spectrum synchronization signal, and wherein the transmission-synchronization module is further configured to determine time synchronizations between the reception times for each of the cardiovascular sensors based on the signal generation time.

19. The system of claim 15, wherein:
the electromagnetic-spectrum signal generator is further configured to:
send a second electromagnetic-spectrum synchronization signal different from the first-mentioned electromagnetic-spectrum synchronization signal; and
the transmission-synchronization module is further configured to:
receive, from a third of the cardiovascular sensors, a second electromagnetic-spectrum synchronization signal mark; and
determine a second time synchronization between the third cardiovascular sensor and the two or more cardiovascular sensors based on the second electromagnetic-spectrum synchronization-signal mark and signal generation times of the electromagnetic-spectrum synchronization signal mark and the second electromagnetic-spectrum synchronization signal mark.

20. The system of claim 19, wherein the first-mentioned and the second electromagnetic-spectrum synchronization signals have different wavelengths and the transmission-synchronization module is further configured to determine which of the different wavelengths is received from each of the cardiovascular sensors.
